(19) United States
(12) Patent Application Publication
(10) Pub. No.: US 2016/0074033 A1
(43) Pub. Date: Mar. 17, 2016

(54) SURGICAL STAPLER

(71) Applicant: Antonio LONGO, Palermo (IT)

(72) Inventor: Federico BILOTTI, Roma (IT)

(21) Appl. No.: 14/783,793

(22) PCT Filed: Apr. 8, 2014
(86) PCT No.: PCT/IB2014/060512

§ 371 Date: Oct. 9, 2015

(30) Foreign Application Priority Data
Apr. 10, 2013 (IT) RM2013A000217

(51) Int. Cl. A61B 17/068; A61B 17/10

(52) U.S. Cl. CPC A61B 17/068; A61B 17/105

(57) ABSTRACT

An anvil assembly is described. The anvil assembly includes including an axis with a proximal and a distal portion, an anvil associated to the distal portion of the axis formed by a central portion and at least a remote rotating portion and a system for reducing the encumbrance of the assembly of the anvil and relative restoring of such encumbrance. This is possible thanks to a half-automatic system specifically activated by the user of the surgical instrument and/or by an automatic system integrated in the system for opening and closing the surgical instrument without the need of activating any specific mechanism. The reduction and the restoring of the anvil size is an advantage during the insertion and the removal of the anvil into and from the organ of the patient.

### TECHNICAL FIELD OF THE INVENTION

The present invention has as subject an anvil assembly for a surgical stapler of the type suitable for applying fasteners in a tissue.

More specifically, the invention relates to a stapler of circular type, in particular suitable to be used in a hollow organ for implementing anastomosis.

### BACKGROUND

In the art several types of surgical instruments for applying suturing elements to a tissue are known, in particular for inserting so-called “fasteners”. The suturing of two extracts or fragments of tissue by means of the latter is performed by means of devices known as staplers.

The surgical staplers generally comprise a cartridge suitable to eject one or more fastener and a so-called “anvil”, or contrast, arranged on the opposite side of the tissue to be sutured with respect to the cartridge. The fasteners indeed abut against the anvil to implement the suture. Upon use, then, the surgeon arranges the two portions of the tissue to be sutured between the cartridge and the anvil and drives the instrument to eject the fasteners, thus determining the penetration of the latter into the two portions of the tissue to be joined.

Both so-called linear staplers, applying one or more rows of fasteners, and so-called circular staplers, applying one or more concentric lines of fasteners, are known. The circular staplers are particularly suitable to be used for the anastomosis of tubular hollow organs, and specifically the intestine and the oesophagus.

In a rather common surgical procedure, known as “end-to-end anastomosis”, after the removal of a intestine tract (needed in case of pathological conditions such as for example a tumour), the remaining intestine ends are joined by using a circular stapler. To this purpose, a fragment of intestine is fastened to the anvil and the other fragment is kept around the cartridge. At this point the anvil is brought near the cartridge, by acting on a knob for closing the stapler. When cartridge and anvil abut one onto the other one, the fasteners are ejected, by penetrating the fragments of tissue and by abutting onto the anvil. During such procedure, generally a circular blade eliminates the superfluous tissue exceeding at the suturing line, by guaranteeing a suitable passage opening and by also allowing the surgeon to check the suturing efficiency. The anvil acts as abutment/end-stroke means even for such blade.

A similar procedure is performed even in the treatment of haemorrhoids, wherein the removal of a whole tract of intestine and therefore the formation of two fragments is not provided, on the contrary the removal of the rectal mucous tissue and the restoring of a passage opening are provided. Even in this case the exceeding tissue is kept between the anvil and the cartridge of a circular stapler and a blade performs the removal of the exceeding tissue simultaneously to the suturing of the remaining portions.

The two types of just described interventions, that is with and without formation of intestine fragments, are schematically shown in FIGS. 1A and 1B.

A drawback of the just described known circular staplers is that the anvil, during the instrument extraction, can meet a certain resistance at the level of suturing line bearing the fasteners. Any forcing during such extraction risks of compromising the integrity of the suturing, which has just been performed and thus results to be more vulnerable. On the contrary, performing a cut nearest to the suturing line in order to obtain a wider passage necessarily makes the sutured region less resistant.

Similar drawbacks can be also found even upon inserting the anvil into the hollow organ or however into the region to be sutured.

A possible solution to facilitate the removal of the stapler after the intervention is provided in U.S. Pat. No. 4,505,272, describing an instrument which can be reduced into four portions. However, the structure of the proposed stapler results to be rather complex and expensive to be produced.

An additional solution is subject of US 2012/0234890. In such case, the base of the anvil is formed by height revolving planar segments, allowing the anvil itself to assume an expanded configuration and a configuration of reduced encumbrance. However, the plurality of joints of such structure can result to be complex.

### SUMMARY OF THE INVENTION

The technical problem placed and solved by the present invention is then to provide an anvil assembly and a relative surgical stapler, in particular of circular type, allowing to obviate the drawbacks mentioned above with reference to the known art.

Such problem is solved by an assembly for surgical stapler according to claim 1 and by a stapler according to claim 14.

Preferred features of the present invention are subject of the depending claims.

In the present context the terms “distal” and “proximal” will be used according to the common use in the field to designate portions respectively farer than, or nearer to, a user in the described configuration.

The anvil assembly of the invention can be inserted and/or removed from the surgical site in a configuration of minimal encumbrance and therefore without compromising the safety of the surgical intervention and the health of the patient.

Furthermore, the anvil assembly of the invention and the stapler wherein it is inserted are suitable to an easy and inexpensive production, with a simple and then intrinsically reliable mechanical structure.

The stapler of the invention is particularly suitable for procedures of anastomosis in hollow organs and in case of haemorrhoids or intestinal prolapse (so-called PPH interventions—Procedure for Prolapse and Hemorrhoids).

The main advantage of the invention consists in facilitating and making atraumatic the insertion of the instrument, especially when the proximal intestinal segment to be anastomosed has a smaller size than the distal segment, typical event in the colorectal anastomoses.

In particular, the invention provides an anvil assembly for surgical stapler bearing a main body and a pair of winglet-like revolving portions, suitable to close onto the main body to assume the above-mentioned configuration of minimal encumbrance. In the open configuration, the anvil assembly, instead, has its maximum encumbrance and it can act efficiently as abutment means of the fasteners and of the blade during the suturing procedure.

The winglet-like portions are directly hinged onto the main body, the latter bearing a distal profile for the penetration into an organ or tissue which is point-shaped.

In an advantageous embodiment a mechanism for driving the above-mentioned revolving portions is provided, based upon a contrast spring or upon an equivalent elastically deformable element, able to exert an opening stress on such portions.

Other advantages, features and use modes of the present invention will result evident from the following detailed description of some embodiments, shown by way of example and not for limitative purposes.

### BRIEF DESCRIPTION OF THE FIGURES

The figures of the enclosed drawings will be referred to, wherein:

FIGS. 1A and 1B, already shown above, illustrate schematically a surgical procedure of anastomosis in a hollow tubular cable, in particular the intestine or oesophagus, and a procedure for suturing haemorrhoids, respectively;

FIG. 2 shows a perspective view of a first preferred embodiment of the anvil assembly of the present invention, in a operative suturing configuration wherein it is comprised in a circular surgical stapler;

FIG. 2A shows an enlarged view of a portion of the assembly of FIG. 2;

FIG. 3 shows a perspective view of the anvil assembly of FIG. 2 in a configuration of minimal encumbrance;

FIG. 4 shows an exploded view of the anvil assembly of FIG. 2;

FIG. 5 shows another exploded view of the anvil assembly of FIG. 2, wherein even additional components are represented with respect to those of FIG. 4;

FIGS. 6A and 6B show each one a view in longitudinal section of the anvil assembly of FIG. 2, in the configuration of minimal encumbrance and in the suturing operative configuration, respectively;

FIG. 7 shows a perspective view of a second preferred embodiment of the anvil assembly of the present invention, in a configuration of minimal encumbrance;

FIG. 8 shows an exploded view of the anvil assembly of FIG. 7;

FIG. 9 shows a perspective view of a third preferred embodiment of the anvil assembly of the present invention, in a configuration of minimal encumbrance;

FIG. 10 shows an exploded view of the anvil assembly of FIG. 9; and

FIGS. 11A and 11B show a side view an a view in longitudinal section, respectively, of the anvil assembly of FIG. 9, in an intermediated configuration between the one of minimal encumbrance and the suturing operative one.

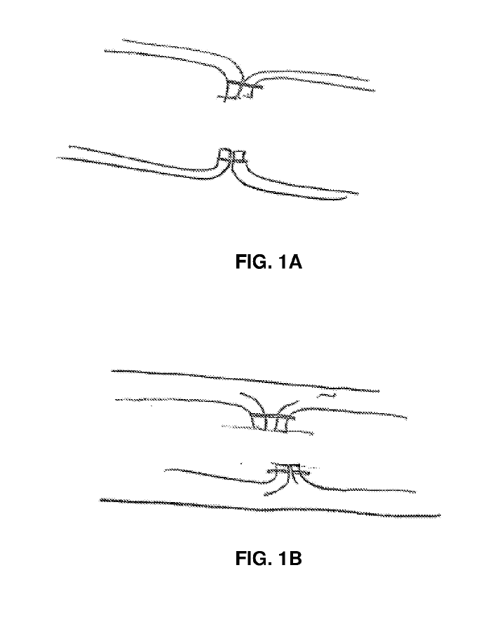

### DETAILED DESCRIPTION OF PREFERRED EMBODIMENTS

Several embodiments and variants of the invention will be described hereinafter and this with reference to the above shown figures.

Analogous components are designated in the several figures with the same reference numeral.

In the following detailed description, additional embodiments and variants, with respect to embodiments and variants already treated in the same description, will be illustrated only in the differences with respect to what already illustrated.

Furthermore, the different embodiments and variants described hereinafter are subjected to be used in combination, where they are compatible.

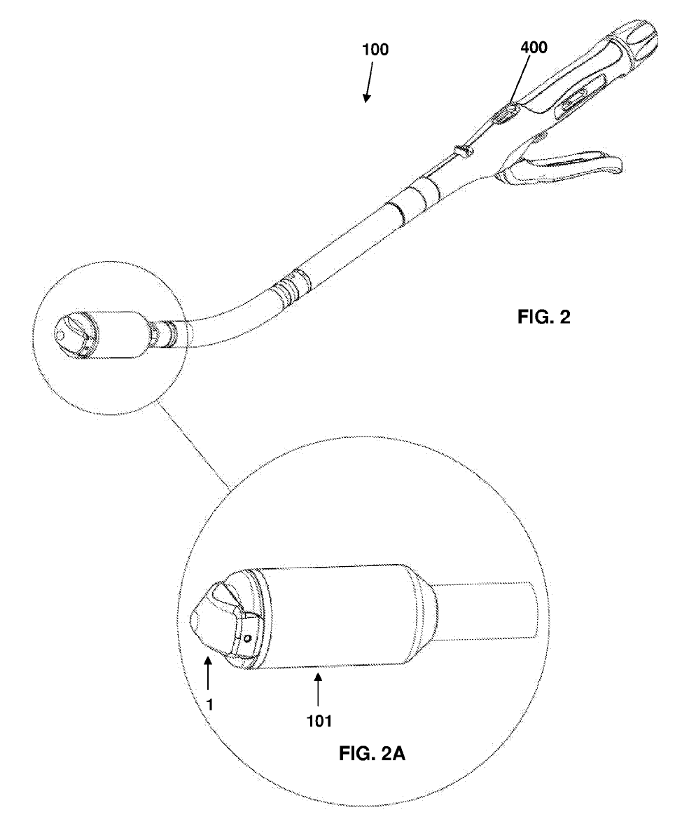

By firstly referring to FIGS. 2 and 2A, a surgical stapler of circular type according to a preferred embodiment of the invention is designated as a whole with 100. The stapler 100 is suitable in applying suturing fasteners to a tissue, in particular a hollow organ for performing anastomosis or for removing portions of the tissue itself, in particular haemorrhoids.

The stapler 100 comprises an anvil assembly according to a first preferred embodiment of the invention, designated as a whole with 1.

The stapler 100 also comprises a cartridge 101, apt for ejection of the fasteners for their penetration into the tissue.

As in the known staplers, the overall arrangement is such that the anvil assembly 1 and the cartridge 101 are suitable to assume a suturing operative configuration, shown in FIGS. 2 and 2A, wherein they are brought near and in particular they abut one onto the other one. In such configuration, the anvil assembly 1 acts as contrast/abutment in the applying of fasteners into the tissue and even as end-stroke means for a cutting blade, indeed as already illustrated in the introduction with reference to the known systems.

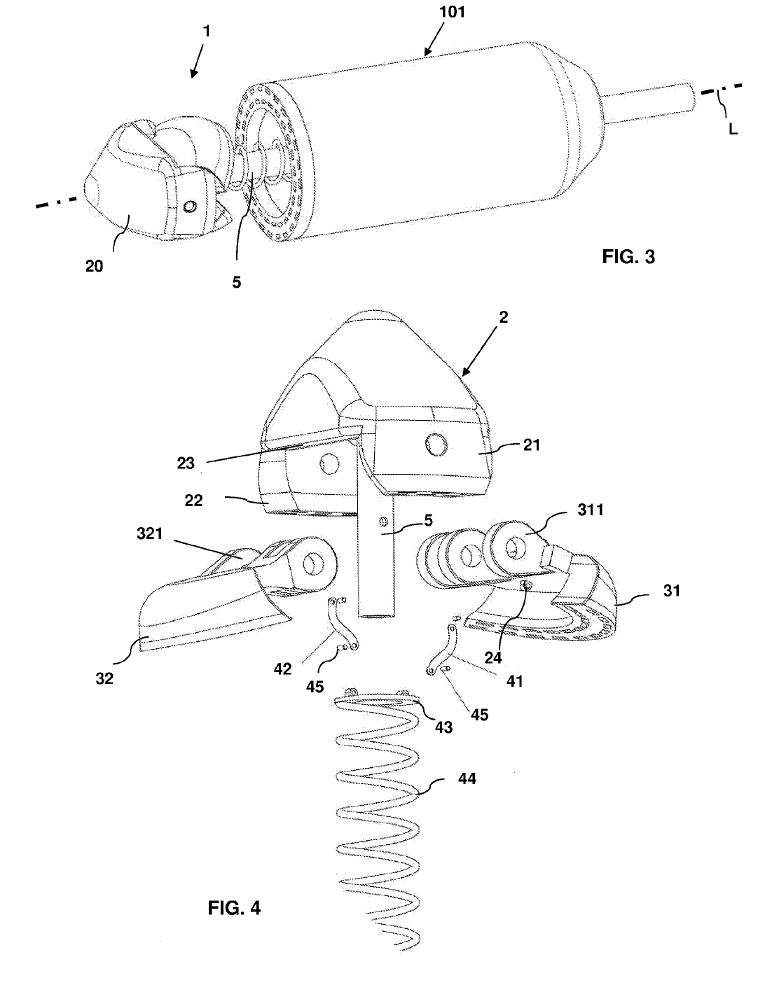

By also referring to FIGS. 3, 4 and 5, the anvil assembly 1 comprises an anvil-like main body 2. In the present embodiment, such main body 2 has a distal profile 20 for the insertion into an organ which is substantially shaped like an arrow point or however pointed.

The main body 2 has a longitudinal axis L even defining a longitudinal axis of the assembly 1 and of the cartridge 101.

The main body 2 comprises a pair of portions 21 and 22 projecting proximally and substantially shaped like a fixed winglet. In the present example, the projecting portions 21 and 22 are arranged on transversally opposed portions with respect to the longitudinal axis L.

The main body 2 is integral to a stem or shaft 5, extending proximally too and substantially according to the longitudinal axis L.

A pair of winglets, respectively 31 and 32, is connected to the main body 2, mobile with respect to the main body. Advantageously, each winglet 31, 32 is rotatably connected directly to the main body 2. In particular, each winglet 31, 32 has a rotational freedom degree with respect to the main body 2 according to a rotation axis which, in the present example, is orthogonal or substantially orthogonal to the longitudinal axis L and, generally, to the insertion direction of the anvil assembly 1 in the patient body.

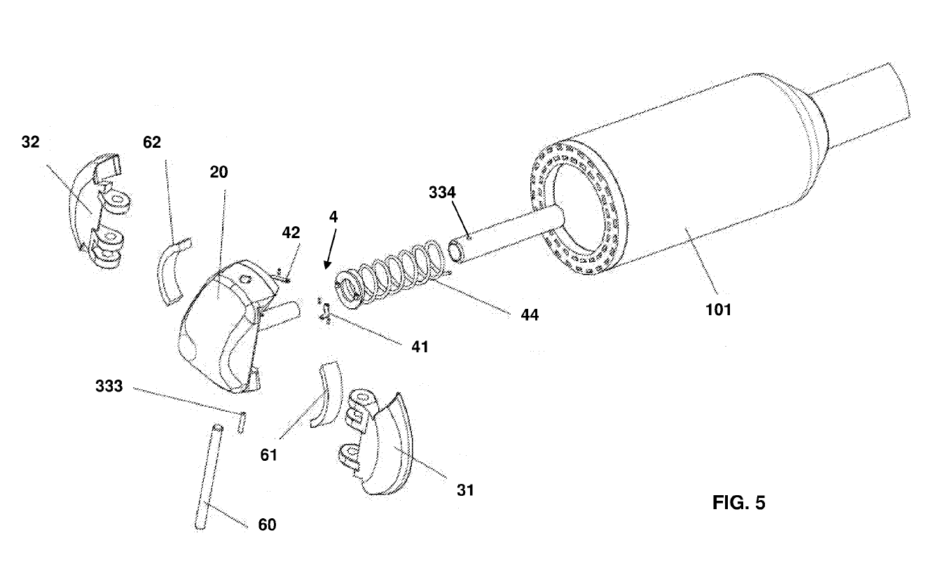

The above-mentioned revolving connection can be implemented by means of connection means known on itself. In particular, in the present example a flange 311, 321 is provided, integral to each winglet and one or more respective pins (one of which represented by way of example in FIG. 5 and designated with 60) associated to the main body 2 and received each one in a suitable seat of a respective flange 311, 312. The longitudinal axis of such or each pin defines the rotation axis of the respective winglet.

Preferably, the configuration is such that said or each winglet 31, 32 has a rotation interval of about 90 degrees.

Still in the present example, the winglets 31 and 32 are arranged on transversally opposite sides of the main body 2 with respect to the longitudinal axis L, so that a fixed winglet 21, 22 is alternated to a revolving winglet 31, 32 along the cross section of the main body 2, that is on the cross periphery of the main body.

As it is better appreciated from the comparison between FIG. 2A and FIG. 3, the rotating capability of the winglets 31 and 32 makes that the assembly 1 can assume a first configuration of minimal encumbrance, shown in FIG. 3, wherein the winglets 31 and 32 are rotated inwards, that is towards the longitudinal axis L and therefore in the proximal direction, and a suturing operative configuration, shown in FIG. 2A, wherein the winglets 31 and 32 are rotated outwardly, that is deployed in the distal direction, and suitable to abut onto the cartridge 101.

The end-stroke of the winglets 31 and 32 in the above-mentioned operative configuration can be defined by corresponding abutment surfaces of the main body 2, one of which is designated with 23 in FIG. 4 by way of example.

The anvil assembly 1 comprises then means for driving the mobile winglets 31 and 32, apt to cause the passage from said configuration of minimal encumbrance to said operative configuration and viceversa, which means is designated as a whole with 4.

In the present advantageous embodiment, the diving means 4 is based upon a coupling between the winglets 31 and 32 and an elastic contrast means, in particular a compression helical spring 44 arranged coaxially to the stem 5.

Such coupling is obtained by means of shaped driving elements 41 and 42, preferably with elongated shape. Each element 41, 42 is connected at a first end thereof to a respective winglet 31, 32 and at the other end to the helicoidal spring 44. To this purpose, seats or connection elements 24 for the driving elements 41 and 42 are obtained on the winglets 31 and 32.

Still in the present arrangement, the above-mentioned connections between the ends of the driving elements 41 and 42 and the respective winglets 31 and 32 and the contrast element 44 are revolving connections implemented, for example, by means of small pins, one thereof designated by way of example with 45 in FIG. 3.

In the present example, each driving means 41, 42 is substantially shaped like a stem with longitudinal profile having one or more loops.

Still in the present embodiment, the driving means 41 and 42 are connected to the elastic contrast element 44 by means of interposing a ring 43. The latter is substantially arranged centred on the longitudinal axis L. Therefore, the loop-like profile of the driving means 41 and 42 allows the latter to project laterally outwardly with respect to the longitudinal axis L.

The ring 43, in turn, is fastened at an arm or a distal turn of the compression spring 44.

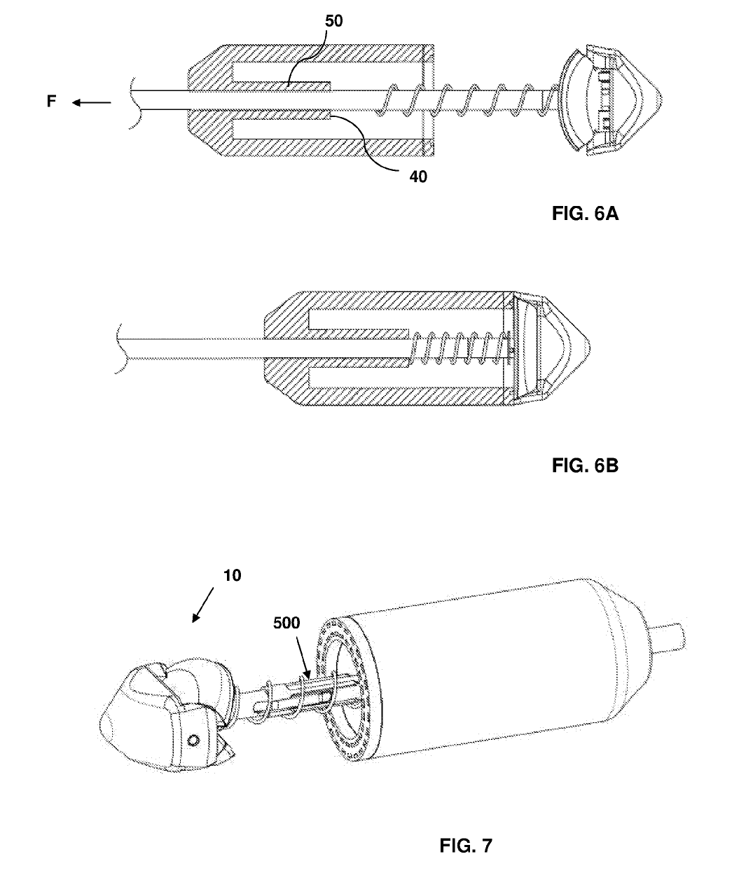

With reference also to FIGS. 6A and 6B, in the herein described embodiment the spring 44 is kept between the winglets 31 and 32 and an abutment surface 40 obtained inside the cartridge 101.

Inside the cartridge 101 a sliding seat 50 for the stem 5 is also obtained, indeed guiding the latter in a substantially translatory motion along the direction of the longitudinal axis L.

Upon making the stem 5 to slide within the seat 50 in proximal direction—that is by pulling the stem 5 towards the user in the direction of the arrow F of FIG. 6A—the main body 2 and the mobile winglets 31 and 32 associated thereto slide integrally to the stem itself. When the proximal end of the spring 44 abuts onto the abutment surface 40, the additional sliding of the stem 5 produces a compression of the spring itself, which then exerts a contrast action on the winglets 31 and 32 by means of the driving means 41 and 42 connected thereto. Such contrast action makes that the driving means 41 and 42 force the winglets 31 and 32 to open outwardly, by reaching the maximum opening with end-stroke means on the surfaces 23 of the main body 2.

In such motion bringing the mobile winglets 31 and 32 to deploy, the driving means 41 and 42 act then each one like a kind of connecting rod. In fact, the longitudinal ends of each element 41, 42 are connected to a sliding element (the spring 44) and to a revolving element (the respective winglet 31, 32), respectively.

Simultaneously or shortly after reaching the above-mentioned deployed configuration, the main body 2 abuts onto the cartridge 101 to carry out its own anvil function with respect to the fasteners ejected by the cartridge itself. Such operative configuration is shown in FIG. 6B.

Of course, the opposite motion of the stem, that is the sliding thereof in the distal direction, produces the restoration of the configuration of minimal encumbrance shown in FIG. 6A.

The sliding of the stem 5 according to the above-mentioned modes can be activated manually by the surgeon by acting on a control or knob 400 of the stapler 100. Such control or knob 400 is shown schematically in FIG. 2 and preferably arranged at a handle portion of the stapler itself.

Alternatively or in association with the manual activation, automatic activation means of the stem 5 can be provided, apt to control the passing of the anvil assembly 1 from the configuration of minimal encumbrance to the operative configuration and/or viceversa. Preferably, such automatic activation is obtained depending upon the relative position, brought near or remote, of the anvil assembly 1 with respect to the cartridge 101. In particular, the closure of the winglets 31 and 32 can be controlled automatically when the main body 2 is moved away from the cartridge 101. Similarly, the opening of the winglets 31 and 32 can be activated automatically when the user brings the main body 2 near the cartridge 101.

Embodiment variants can provide a different arrangement of portions and in particular an elastically deformable means, different from the above-considered spring.

An embodiment variant can further provide that the driving means bases upon a positive coupling between the winglets 31 and 32 and the respective driving elements 41 and 42. In particular, respective cam-like surfaces or profiles can be obtained on the winglets, arranged at an inner face of the winglet, that is the face directed towards the longitudinal axis L. The engagement of such surfaces by the respective driving elements 41, 42 moved in the distal direction determines the opening out of the winglets 31 and 32 themselves.

In such case, then, the described positive coupling allows transforming the longitudinal motion of the stem 5 into the revolution one of the winglets 31 and 32.

An additional variant can even provide one single portion like a mobile winglet instead of the pair of the above described winglets.

Going back to FIG. 5, two acknowledgment or feedback elements are represented, designated with 61 and 62, respectively, and preferably made of plastic. Each one of the feedback elements 61, 62 is integral to the main body 2 and arranged on the latter at an inner face of a respective mobile winglet 31, 32. Each feedback element 61, 62 is suitable indeed to provide a (in particular auditory) acknowledgment of carried-out cut after driving the blade of the stapler (which blade is not represented in the figures, being known on itself).

Even these elements are already known in the art and thus will not further dwell upon the description thereof.

FIG. 5 also shows a retaining element 333, with elongated shape. It is suitable to be inserted in a hole 334 of the stem 5 and prevents the spring 44 from falling into the instrument by moving away from the main body 2, when the latter is far from the cartridge 101 and the spring 44 is released. Such element then acts as proximal acknowledgment or end-stroke means for the spring itself.

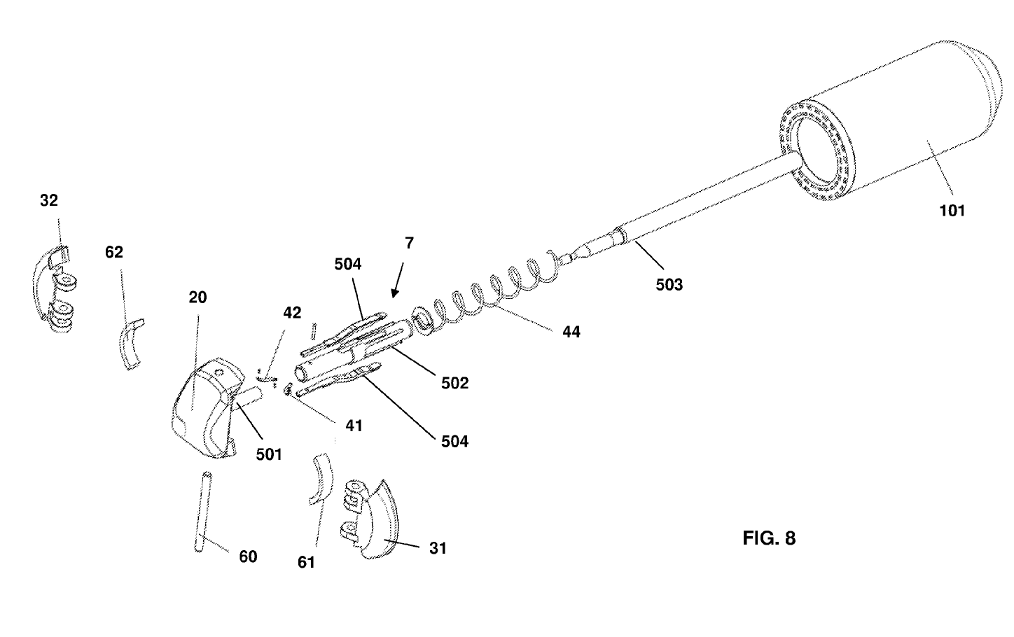

FIGS. 7 and 8 refer to a second preferred embodiment of the anvil assembly of the invention, herein designated as a whole with 10.

With respect to the already described first embodiment and related variants, the herein considered assembly is removable with respect to the remaining portion of the stapler. To this purpose, the anvil assembly 10 indeed has means for the removable connection to the surgical stapler, designated as a whole with 7 and arranged at the stem, herein designated with 500.

In the represented exemplifying arrangement, the stem 500 is implemented in three portions, 501, 502 and 503 respectively. The first portion 501, or distal portion, is integral to the main body 2 of the anvil assembly 10. The second portion 502 is an intermediate portion for the connection of the distal portion 501 to the third proximal portion 503, the latter associated to the remaining portion of the surgical stapler. The three portions 501, 502 and 503 are made removably integral one to the other one by means of quick engagement or equivalent means, in case based upon different types of shape coupling.

In particular, the coupling shown in FIG. 8 includes two bending elements 504, for example substantially shaped like a winglet. Such elements 504 are stiff in the distal portion thereof and flexible in the proximal portion thereof. This flexibility allows the elements 504 to engage the third proximal portion 503, by hooking it to the intermediate portion 502 and by keeping it integral to the latter as long as a force higher than the resistance of the elements 504 is not applied to the means 7 in order to release the two portions 501 and 502 from the proximal portion 503. In the present example, even the portions 501 and 502 engage removably one in the other one.

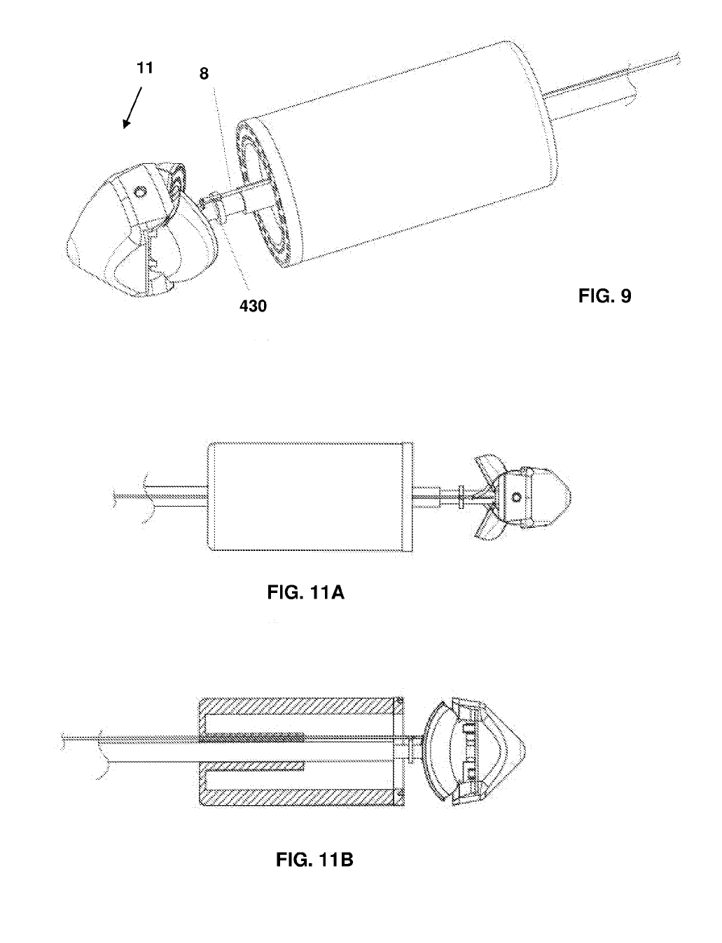

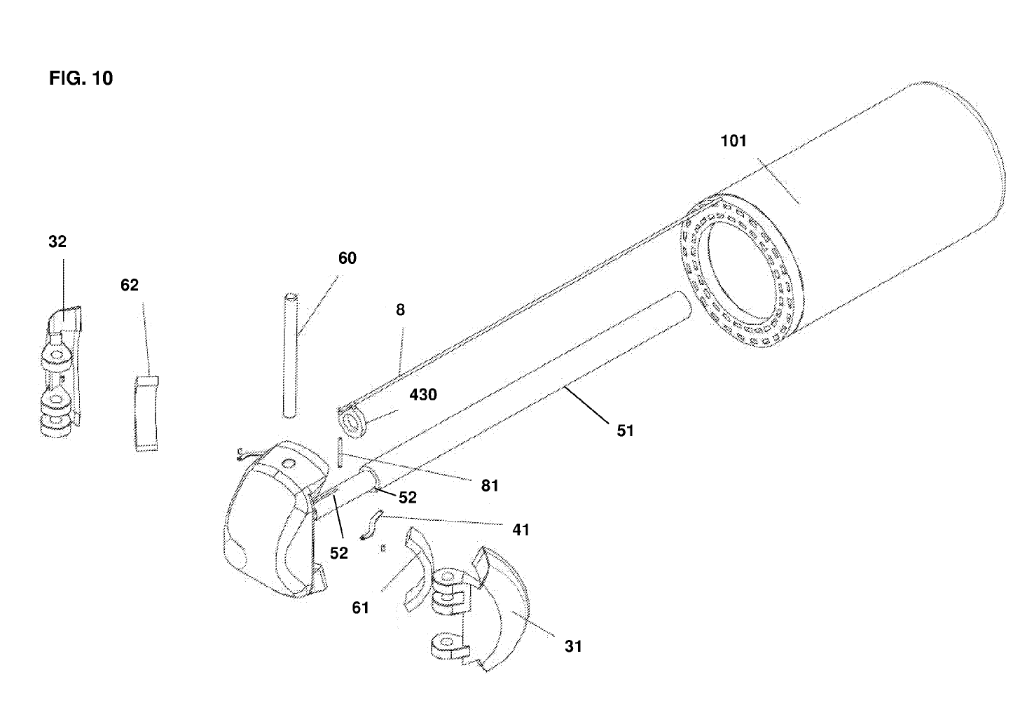

FIGS. 9 to 11 B refer to a third preferred embodiment of the anvil assembly of the invention, herein designated as a whole with 11.

With respect to the preceding embodiments and variants, the anvil assembly 11 does not provide any contrast element and the activation of the driving means 41 and 42 is only manual. To this purpose, a shaft 8 is provided thereto a ring 430 is made integral. The latter is arranged coaxially on the stem of the anvil assembly 11, which stem is designated with 51. The ring 430 guides a sliding motion of the shaft 8 on the stem 51 itself. The latter has also a proximal end-stroke means 52 for the ring 430, preferably in the form of an abutment surface, in this case an annular surface.

An elongated guiding element 81 is made integral, distally, to the shaft 8, which element is sliding within a longitudinal slot 53 of the stem 51, integrally to the shaft 8.

At opposed cross ends of the element 81, in particular on opposite sides with respect to the stem 51, the already shown driving means 41 and 42 are connected on the element 81 itself.

The operating modes of the assembly 11 for the rest can be analogous to the ones already described above with reference to the previous embodiment and related variants.

The present invention has been sofar described with reference to preferred embodiments. It is to be meant that other embodiments belonging to the same inventive core may exist, as defined by the protection scope of the herein below reported claims.

1. An anvil assembly,
adapted for use in a surgical stapler, wherein the anvil assembly acts as abutment when applying suture fasteners to a tissue,
in particular in anastomosis interventions or interventions of suturing hollow tubular organs,
which anvil assembly comprises:
an anvil-like main body bearing a point-like distal profile suitable for the insertion into an organ;
a pair of mobile winglets, each one directly connected to said main body and rotatable with respect to said main body between a configuration of minimal encumbrance of the anvil assembly and an operative suturing configuration in which the winglet is deployed and suitable to abut on a cartridge of the stapler;
a driver for driving said winglets, confiqured to cause passing from said configuration of minimal encumbrance to said operative configuration and/or viceversa; and
a pair of fixed winglets, integral to said main body so that one fixed winglet is alternated to a mobile winglet along the cross periphery of the main body.

2. The anvil assembly according to claim 1, wherein said mobile winglets are arranged at opposite parts of said main body.

3. The anvil assembly according to claim 1, wherein the configuration is such that each mobile winglet has a rotation interval of about 90 degrees.

4. The anvil assembly according to claim 1, including one single pair of mobile winglets and one single pair of fixed winglets.

5. The anvil assembly according to claim 1, comprising end stroke means for stopping the motion of each mobile winglet, preferably obtained at said main body.

6. The anvil assembly according to claim 1, wherein said driver comprises an elastic contrast element, preferably a compression spring, suitable to force the deployment of each mobile winglet in said operative configuration.

7. The anvil assembly according to claim 1, comprising a stem integral to said main body and preferably comprising an elastic contrast element suitable to force the deployment of each mobile winglet in said operative configuration, wherein said contrast elastic element is arranged at said stem.

8. The anvil assembly according to claim 1, wherein said driver comprises slidable means with respect to said main body and suitable to produce deployment of each mobile winglet in said operative configuration.

9. The anvil assembly according to claim 8, wherein said sliding means comprises a shaft, preferably manually operable by the surgeon.

10. The anvil assembly according to claim 1, wherein said driver comprises at least one driving element, preferably a pair of driving elements, wherein said or each driving element is connected or coupled to a respective mobile winglet and to an elastic contrast element.

11. The anvil assembly according to claim 10, wherein said or each driving element has an oblong shape, preferably having at least one loop, and it is connected or coupled to the respective winglet and to said contrast elastic element at one of its own opposite longitudinal ends.

12. The anvil assembly according to claim 10, wherein said or each driving element is rotatably connected to the respective mobile winglet and to said contrast elastic element.

13. The anvil assembly according to claim 10, wherein said or each driving element has a connecting-rod-like arrangement.

14. The anvil assembly according to claim 1, wherein said driver comprises shape-coupling means with each winglet, which shape-coupling means preferably has one or more cam-shaped surfaces, the latter preferably obtained on each winglet.

15. The anvil assembly according to claim 14, wherein said driver comprises at least one shaped element suitable to engage a respective cam-shaped surface.

16. The anvil assembly according to claim 1, comprising removable connecting means for a removable connection to the surgical stapler, preferably arranged at a stem associated to said main body.

17. A surgical stapler, in particular of circular type, suitable for use in anastomosis interventions or interventions of suturing hollow tubular organs for the applying of suture fasteners to a tissue, comprising:
the anvil assembly according to claim 1; and
a cartridge adapted for ejection of the fasteners for their penetration into the tissue,
the overall configuration being such that, in said operative suturing configuration, said anvil assembly abuts on said cartridge acting as abutment in the applying of fasteners into the tissue.

18. The surgical stapler according to claim 17, having an abutment surface and wherein said driver of said anvil assembly comprises an elastic contrast element, preferably a compression spring, suitable to force the deployment of each winglet in said operative configuration, which elastic element is interposed between each winglet and an abutment surface.

19. The surgical stapler according to claim 18, wherein said abutment surface is obtained at said cartridge.

20. The surgical stapler according to claim 17, comprising manual activation means of said driver, which activation means is preferably arranged at a handle portion of the stapler itself.

21. The surgical stapler according to claim 17, comprising automatic activation means of said driver, confiqured to command the passing of said anvil assembly from said configuration of minimal encumbrance to said operative configuration and/or viceversa, preferably depending upon the relative position, brought near or remote, of said anvil assembly with respect to said cartridge.
